(19) United States
(12) Patent Application Publication
(10) Pub. No.: US 2005/0033090 A1
(43) Pub. Date: Feb. 10, 2005

(54) Process for the preparation of cinnamaldehyde compounds

(75) Inventors: Thomas Osswald, Basel (CH); Olivier Ruha, Pratteln (CH)

Correspondence Address: ROPES & GRAY LLP, ONE INTERNATIONAL PLACE, BOSTON, MA, 02110-2624, US

(73) Assignee: LymphoSign Inc., Markham (CA)

(21) Appl. No.: 10/880,430

(22) Filed: Jun. 29, 2004

(30) Foreign Application Priority Data
Jun. 30, 2003 (CH) 01149/03

(51) Int. Cl. C07C 45/90

(52) U.S. Cl. 564/305; 568/433

(57) ABSTRACT

A process for the preparation of cinnamaldehyde, α,β-unsaturated cyanoester, and cyanoamide compounds using a Heck reaction is described. Methods for further elaboration of these aldehydes are also provided.

### CROSS REFERENCE TO RELATED APPLICATIONS

This application claims the priority benefit of Swiss application 01149/03, filed on Jun. 30, 2003, the contents of which are incorporated herein by reference in their entirety.

### FIELD OF THE INVENTION

The present invention relates to a process for the preparation of cinnamaldehyde compounds and to the use of the cinnamaldehyde compounds for the preparation of α,β-unsaturated cyanoester and cyanoamide compounds.

### BACKGROUND OF THE INVENTION

A number of compounds have been identified that inhibit abnormal cell proliferation, for example cancer cell growth, and which preferably do not adversely affect normal cell proliferation. These compounds are disclosed in WO 01/79158, WO 03/062190, U.S. Ser. No. 09/834,728, U.S. Ser. No. 10/240,740, U.S. Ser. No. 10/803,607, U.S. 60/556,972, U.S. 60/349,214, U.S. 60/491,109, and U.S. 60/49119 which are hereby incorporated by reference in their entirety. However, there remains a need for an improved synthetic process for the production of these compounds.

### SUMMARY OF THE INVENTION

The present invention provides a process for the preparation of cinnamaldehyde compounds of the general formula (I): 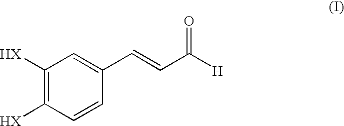
in which X is —O— or —NH—,
which is characterized in that a compound of the general formula (II): 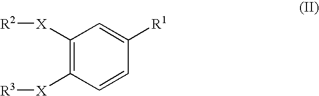
in which
R¹ is a leaving group which is able to react in a Heck reaction as complex-forming leaving group,
X is —O— or —NH—; and
when X is —O—, R² and R³, independently of one another, are trialkylsilyl, (C₁₋₄)-alkyl, (C₁₋₄)-alkenyl, aryl; or R² and R³ together are a divalent protecting group, preferably —C(CH₃)₂—, —CH₂—, —CH₂—CH₂—, —C(O)—C(O)—, or dialkylsilyl, thereby forming a ring; and
when X is —NH—, R² and R³, independently of one another, are trialkylsilyl or alkyloxycarbonyl or phenyloxycarbonyl, or R² and R³ together are —C(O)—C(O)—;
is reacted with a compound of the general formula (III): 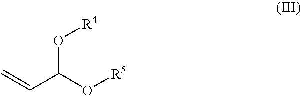
in which
R⁴ and R⁵, independently of one another, are C₁₋₈-alkyl or R⁴ and R⁵ together are a cyclic acetal, preferably, R⁴ and R⁵ together are C₁₋₆alkyl, thereby forming a ring, more preferably R⁴ and R⁵ together are C₂₋₃alkyl, in a Heck reaction, and then the protective groups are removed.

R¹ is leaving group which is able to react in a Heck reaction as a complex-forming leaving group, preferably halogen, trifluoromethanesulphonate [—OS(O)₂CF₃, TfO]; carbonyl halide [—C(O)Hal], nitro, or diazo (N₂⁺); —N₂BF₄; preferably chlorine, bromine or iodine, trifluoromethanesulphonate, or carbonyl chloride [—C(O)Cl]; preferably bromine.

X is preferably —O—.

When X is —O—, R² and R³, independently of one another, are preferably trimethylsilyl, methyl, phenyl, or R₂ and R₃ are together —C(CH₃)₂—, —CH₂—, —CH₂—CH₂—, or dimethylsilyl, thereby forming a ring; more preferably, R₂ and R₃ together are —C(CH₃)₂—, —CH₂—, or —CH₂—CH₂—, and most preferably —CH₂—.

When X is —NH—, R² and R³, independently of one another, are preferably trialkylsilyl or alkyloxycarbonyl, preferably trimethylsilyl or Boc (tert-butyloxycarbonyl).

Alkyloxycarbonyl includesincludes, but is not limited to, isobutyloxycarbonyl, tert-butyloxycarbonyl, tert-amyloxycarbonyl, cyclobutyloxycarbonyl, 1-methylcyclobutyloxycarbonyl, cyclopentyloxycarbonyl, cyclohexyloxycarbonyl, 1-methylcyclohexyl, of which tert-butyloxycarbonyl is preferred.

R⁴ and R⁵, independently of one another, are preferably methyl, ethyl or trimethylsilyl or or R⁴ and R⁵ together are a cyclic acetal, preferably, R⁴ and R⁵ together are C₁₋₆alkyl, thereby forming a ring, more preferably R⁴ and R⁵ together are C₂₋₃alkyl. The compound of the formula (III) is preferably acrolein ethylene acetal.

A preferred embodiment of the reaction according to the invention can be formulated as follows:

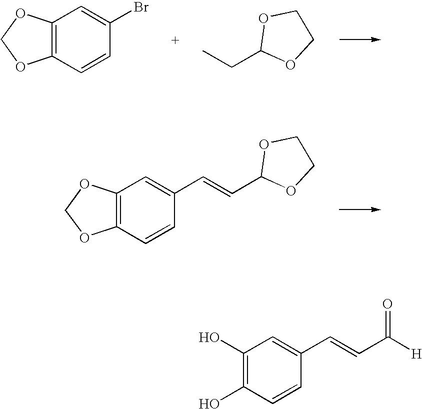

The Heck reaction is known per se. With the reaction according to the invention, in accordance with the above embodiment, of 1-bromo-3,4-(methylenedioxy)benzene with the unsaturated compound acrolein ethylene acetal, a new C—C bond is formed, with the bromine atom serving as leaving group.

The conditions for introducing the protective groups, i.e., for the preparation of the compounds of the general formulae (II) and (III), are known per se (Greene, T. W.; Wuts, P. G. M. Protective Groups in Organic Synthesis, 2^{nd} ed.; Wiley: New York, 1991).

To introduce the protective group in which R², R³, R⁴ and/or R⁵ are trialkylsilyl, i.e., for the silylation of the OH group and/or the NH group, preference is given to using a (alkyl)₃Si(halogen), e.g., (CH₃)₃SiCl, or bistrimethylsilyltrihaloacetamide, bistrimethylsilylacetamide, hexamethyldisilazane and/or bistrimethylurea, preferably bistrimethylsilyltrifluoroacetamide, or a trialkylsilyl trifluoromethanesulphonate, preferably trimethylsilyl trifluoromethanesulphonate. The reaction conditions for the silylation are known per se.

To introduce a protective group in which R² and/or R³ are alkyloxycarbonyl, e.g., tert-butyloxycarbonyl (Boc), the procedure is carried out in a manner known per se, by reacting the precursor of the compound of the general formula (I), which has at least one —NH group, preferably at least one NH₂ group, e.g., with Boc anhydride (Boc-O-Boc) {[(CH₃)₃C—O—C(O)]₂—O} or with Boc carbamate [(CH₃)₃C—O—C(O)—N(C₁₋₄-alkyl)₂]. Such analogous reactions are described in the literature.

The conditions for introducing a protective group in which R² together with R³ are —CH₂—, —C(CH₃)₂—, or —C(O)—C(O)—, thereby forming a ring, are known per se. To introduce —CH₂—, the starting materials are preferably methylal and 1,2-diols. To introduce —C(CH₃)₂—, the starting materials are preferably acetone and analogous compounds. To introduce —C(O)—C(O)—, the starting materials are preferably oxalyl chloride (oxalic acid chloride) or malonyl chloride (malonic acid chloride), most preferablyoxalyl chloride.

The conditions for introducing a protecting group wherein R⁴ and R⁵ together are C₁₋₆alkyl, the starting materials are preferably 1,2-diols. To introduce —CH₂CH₂—, the starting materials are preferably HOCH₂CH₂OH and analogous compounds.

To remove the protective groups, the resulting compound is preferably treated with a suitable acid, for example with hydrochloric acid, formic acid, acetic acid and/or trifluoroacetic acid, preferably with hydrochloric acid or formic acid.

Methods of isolating the compounds of the general formula (I) from the reaction mixture, and of further purifying them are known to the person skilled in the art.

The present invention also provides a process for the preparation of cinnamaldehyde compounds of the general formula (IV) 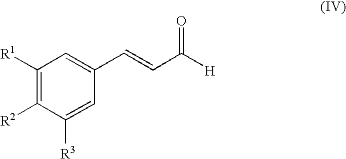
wherein
R¹ and R² are indepedently selected from H, OH, C₁₋₆alkyl, C₁₋₆alkoxy, C₁₋₆alkylCO₂, NH₂, NH-C₁₋₆alkyl, N(C₁₋₆alkyl)(C₁₋₆alkyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, CF₃, OCF₃, heterocyclyl, and halo, or R¹ and R² together represent O-C₁₋₆alkyl-O, thereby forming a ring;
R³ is selected from H, OH, C₁₋₆alkyl, C₁₋₆alkoxy, C₁₋₆alkylCO₂, NH₂, NH-C₁₋₆alkyl, N(C₁₋₆alkyl)(C₁₋₆alkyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, heterocyclyl, halo, and CH₂—S—(CH₂)ₙ Ar;
Ar is an aromatic or heteroaromatic group, unsubstituted or substituted with 14 substituents, independently selected from OH, C₁₋₆alkyl, C₁₋₆alkoxy, NH₂, NH-C₁₋₆alkyl, N(C₁₋₆alkyl)(C₁₋₆alkyl), SH, S-C₁₋₆alkyl, NO₂, CF₃, OCF₃, and halo; and
n is 0 to 4;
comprising reacting a compound of the general formula (II) 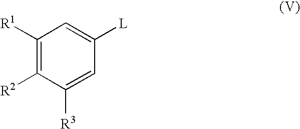
wherein
R¹ and R² are independently selected from H, C₁₋₆alkyl, C₁₋₆alkoxy, C₁₋₆alkylCO₂, NH-C₁₋₆alkyl, N(C₁₋₆alkyl)(C₁₋₆alkyl), NH(C₁₋₆alkyloxycarbonyl), NH(phenyloxycarbonyl), NH(C₁₋₆trialkylsilyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, CF₃, OCF₃, heterocyclyl, and halo, or R¹ and R² together represent O-C₁₋₆alkyl-O (preferably —O—C(CH₃)₂—O— or —OCH₂O—), —C(O)—C(O)—, or dialkylsilyl, thereby forming a ring;
R³ is selected from H, C₁₋₆alkyl, C₁₋₆alkoxy, C₁₋₆alkylCO₂, NH-C₁₋₆alkyl, N(C₁₋₆alkyl)(C₁₋₆alkyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, heterocyclyl, halo, and CH₂—S—(CH₂)ₙ Ar;
L is a leaving group which is able to react in a Heck reaction as complex-forming leaving group;
Ar is an aromatic or heteroaromatic group, unsubstituted or substituted with 1-4 substituents, independently selected from OH, C₁₋₆alkyl, C₁₋₆alkoxy, NH₂, NH-C₁₋₆alkyl, N(C₁₋₆alkyl)(C₁₋₆alkyl), SH, S-C₁₋₆alkyl, NO₂, CF₃, OCF₃, and halo; and
n is 0 to 4;
with a compound of the general formula (III).

The catalyst used in the Heck reaction is preferably chosen from compounds of palladium (Pd). Examples of such palladium compounds are: Pd(0) compounds, such as tris(dibenzylideneacetone)dipalladium chloroform complex, Pd(PPh₃)₄, and Pd(II) compounds, such as PdCl₂, Pd(dppe)₂, [dppe=bis-(1,2-biphenylphosphino)ethane], Pd(dppe)Cl₂, Pd(OAc)₂, Pd(dppe)(OAc)₂, Pd(CH₃CN)₂Cl₂, Pd(PPh₃)₂Cl₂, π-allyl-Pd complexes, preferably π-allyl-Pd chloride dimer. Preference is given to Pd(0) compounds, in particular tris(dibenzylideneacetone)dipalladium chloroform complex. Further catalysts are also Pd/C, Pd/Mg, and palladium which is deposited on diverse substrates. These compounds are known per se and described in the literature.

As is already at times evident from the given examples, the palladium complex can be thermally stabilized using an additional complexing agent, such as 2,2′-bipyridyl or 1,10-phenanthroline. It is likewise possible to use phosphine compounds, such as, for example, triphenylphosphine, tritolylphosphine, DPPM (1,1-bis(diphenylphosphino)methane, DPPE (1,2-bis(diphenylphosphino)ethane, DPPB (1,4-bis(diphenylphosphino)butane, DPPF (1,1′-bis(diphenylphosphino)ferrocene and related compounds known per se.

For the reaction, the solvents which may be used are all common organic anhydrous compounds, such as, for example, toluene, petroleum spirit, hexane, heptane, tert-butyl alcohol, diethyl ether, acetone, benzene, dioxane, tetrahydrofuran, chloroform, dimethylformamide or pyridine. Very generally, the conditions known per se for the Heck reaction can be used.

The present invention further provides a process for the preparation of α,β-unsaturated cyanoester and cyanoamide compounds of the general formula (VI): 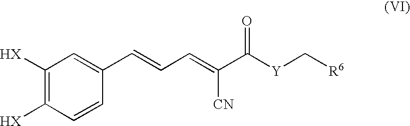
in which
X is —O— or —NH—
Y is —O— or —NH— and
R⁶ is optionally substituted phenyl or phenyl-(C₁₋₄alkyl,
which is characterized in that a compound of the general formula (I) given above is reacted in accordance with Knoevenagel with a compound of the general formula (VII): 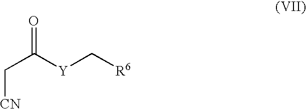
in which Y and R⁶ have the meanings given above. Here, Y is preferably —NH—. R₆ is preferably phenyl.

The reaction according to the invention can be carried out with a high yield. The reaction can also be carried out if the hydroxyl groups or the amino groups of the compound of the formula (VI) are unprotected.

Preference is given to the preparation of the following compounds:
(E,E)-2(benzylamido)-3-(3,4-dihydroxystyryl)acrylonitrile;
(E,E)-2(phenylethylamido)-3-(3,4-dihydroxystyryl)acrylonitrile;
(E,E)-2(phenylpropylamido)-3-(3,4-dihydroxystyryl)acrylonitrile;
(E,E)-2(2,4-dihydroxybenzyl)-3-(3,4-dihydroxystyryl)acrylonitrile;
(E,E)-2(benzylamido)-3-(3,4-diaminostyryl)acrylonitrile.

The present invention also provides a process for the preparation of α,β-unsaturated cyanoester and cyanoamide compounds of the general formula (VIII) 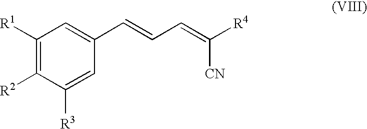
wherein
R¹ and R² are independently selected from H, OH, C₁₋₆alkyl, C₁₋₆alkoxy, C₁₋₆alkylCO₂, NH₂, NH-C₁₋₆alkyl, N(C₁₋₆alkyl)(C₁₋₆alkyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, CF₃, OCF₃, heterocyclyl, and halo, or R¹ and R² together represent O-C₁₋₆alkyl-O (preferably —O—C(CH₃)₂—O— or —OCH₂O—), —C(O)—C(O)—, or dialkylsilyl, thereby forming a ring;
R³ is selected from H, OH, C₁₋₆alkyl, C₁₋₆alkoxy, C₁₋₆alkylCO₂, NH₂, NH-C₁₋₆alkyl, N(C₆alkyl)(C₁₋₆alkyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, heterocyclyl, halo and CH₂—S—(CH₂)ₙ Ar;
R⁴ is selected from C(X)R⁵, SO₃Ar, SO₂Ar, SO₂(C₁₋₆alkyl), NH₂, NH-C₁₋₆alkyl, N(C₁₋₆alkyl)(C₁₋₆alkyl), P(O)(OH)₂, P(O)(OC₁₋₆alkyl)₂, and C(NH₂)═C(CN)₂;
X is selected from O, S, NH, and N-C₁₋₆alkyl;
R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, (OCH₂CH₂)ₚOCH₃, NHNH₂, NHC(O)NH₂, NHC(O)C₁₋₆alkoxy, N-morpholino, and N-pyrrolidino;
Ar is an aromatic or heteroaromatic group, unsubstituted or substituted with 1-4 substituents, independently selected from OH, C₁₋₆alkyl, C₁₋₆alkoxy, NH₂, NH-C₁₋₆alkyl, N(C₁₋₆alkyl)(C₁₋₆alkyl), SH, S-C₁₋₆alkyl, NO₂, CF₃, OCF₃, and halo;
n is 0 to 4; and
p is 1-4;
comprising reacting a compound of the general formula (VIII) in accordance with Knoevenagel with a compound of the general formula (IX) 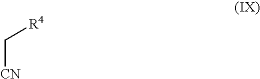
wherein
R⁴ represents C(X)R⁵, SO₃Ar, SO₂Ar, SO₂(C₁₋₆alkyl), NH₂, NH-C₁₋₆alkyl, N(C₁₋₆alkyl)(C₁₋₆alkyl), P(O)(OH)₂, P(O)(OC₁₋₆alkyl)₂, and C(NH₂)═C(CN)₂;
X is selected from O, S, NH, and N-C₁₋₆alkyl;
R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, (OCH₂CH₂)ₚOCH₃, NHNH₂, NHC(O)NH₂, NHC(O)C₁₋₆alkoxy, N-morpholino and N-pyrrolidino;
Ar is an aromatic or heteroaromatic group, unsubstituted or substituted with 14 substituents, independently selected from OH, C₁₋₆alkyl, C₁₋₆alkoxy, NH₂, NH-C₁₋₆alkyl, N(C₁₋₆alkyl)(C₁₋₆alkyl), SH, S-C₁₋₆alkyl, NO₂, CF₃, OCF₃ and halo;
n is 0 to 4; and
p is 1-4.

The term “in accordance with Knoevenagel” or a “Knoevenagel reaction” is known in the art and encompasses reactions wherein an activated methylene and an aldehyde or ketone are treated with base to afford an olefin.

The term “activated methylene” is art-recognized and includes methylene groups (CH₂) with a pKa between 10 and 20, preferably between 10 and 15. This can be accomplished by functionalization of the methylene group with at least one electron withdrawing group, wherein the term electron withdrawing group includes, but is not limited to, carboxylic ester, carboxylic acid, nitrile, nitro, or carbonyl.

The term “heteroatom” as used herein means an atom of any element other than carbon or hydrogen. Preferred heteroatoms are nitrogen, oxygen, phosphorus, and sulfur.

The term “heterocycle”, “heterocyclic group”, or “heterocyclyl” is art-recognized and includes substituted or unsubstituted non-aromatic 3- to 10-membered ring structures, more preferably 3- to 7-membered rings, whose ring structures include one to four heteroatoms. The term terms “heterocycle”, “heterocyclic group”, or “heterocyclyl” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

In preferred embodiments, R¹, R² and R³ are each independently selected from H, OH, OCH₃, CH₃CO₂, NH₂, N(CH₃)₂, and NO₂. In most preferred embodiments, R¹, R² and R³ are each independently selected from H, OH, and OCH₃, provided that at least one group is other than hydrogen.

In preferred embodiments, R⁴ is selected from C(X)R⁵, SO₂Ar, SO₂(C₁₋₆alkyl), and C(NH₂)═C(CN)₂. More preferably, R⁴ is C(X)R⁵. In preferred embodiments, X is O or S and R⁵ is selected from NH₂, OH, NH(CH₂)ₚAr, (CH₂)ₚOH and C₁₋₄alkoxy, (where p is 1-3). Most preferred, are compounds wherein X is 0 and R⁵ is selected from NH2, OH, NH(CH₂)ₚAr, NH(CH₂)ₚOH and OCH₃, (where p is 1-2).

The present invention includes compounds wherein Ar is an unsubstituted or substituted aryl and/or heteroaryl group. In preferred embodiments, Ar is an unsubstituted phenyl group or phenyl group substituted with 1-2 substituents optionally selected from OH, C₁₋₄alkyl, C₁₋₄alkoxy, NH₂, NH-C₁₋₄alkyl, N(C₁₋₄alkyl)(C₁₋₄alkyl), SH, S-C₁₋₄alkyl, NO₂, CF₃, OCF₃ and halo. In more preferred embodiments, Ar is an unsubstituted phenyl group or phenyl group substituted with 1-2 substituents optionally selected from OH, OCH₃, NH₂, NHCH₃, N(CH₃)₂, SH, SCH₃, CF₃, OCF₃ and halo.

In the most preferred embodiments of the present invention, a compound having one of the following structures is prepared

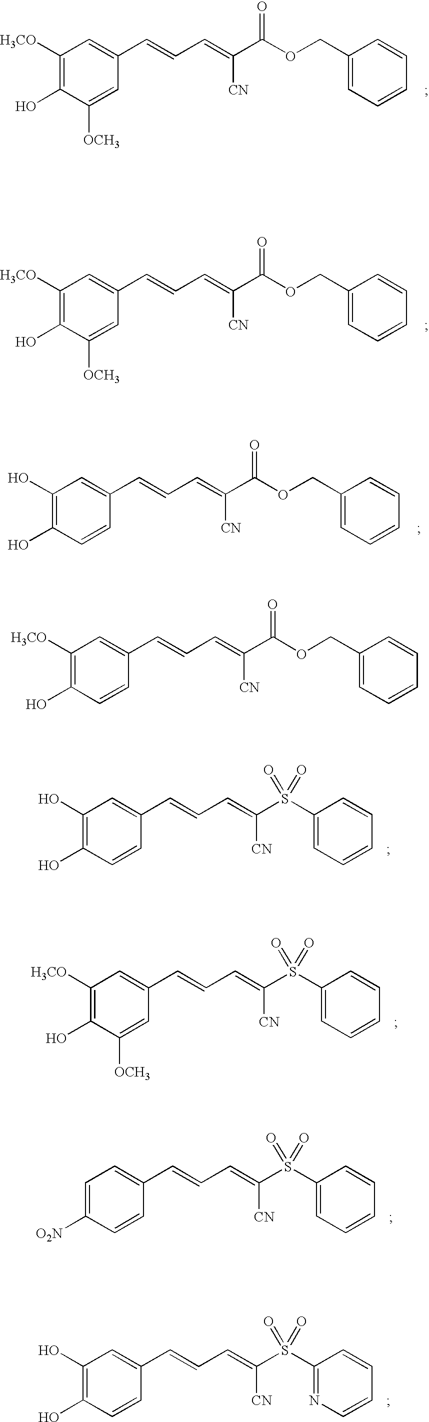

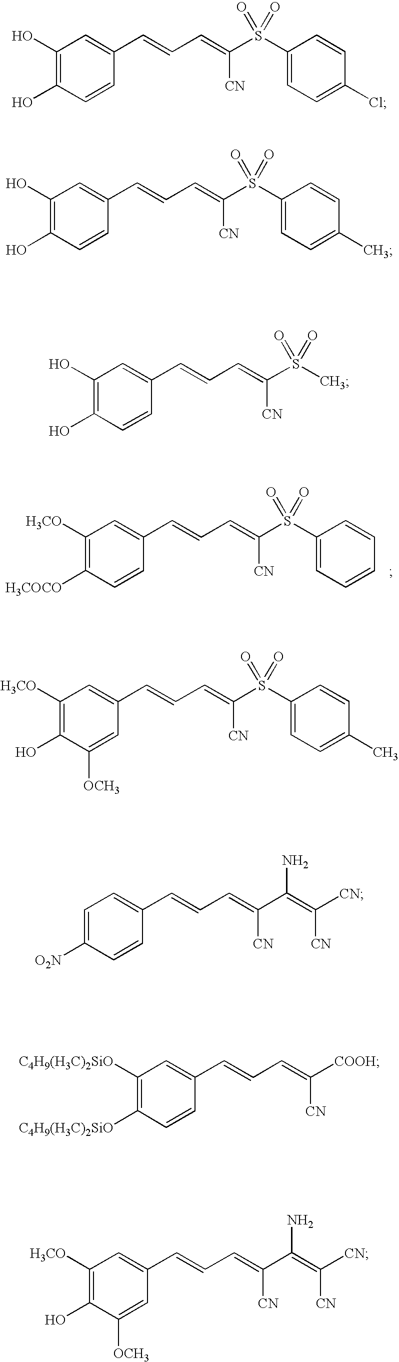

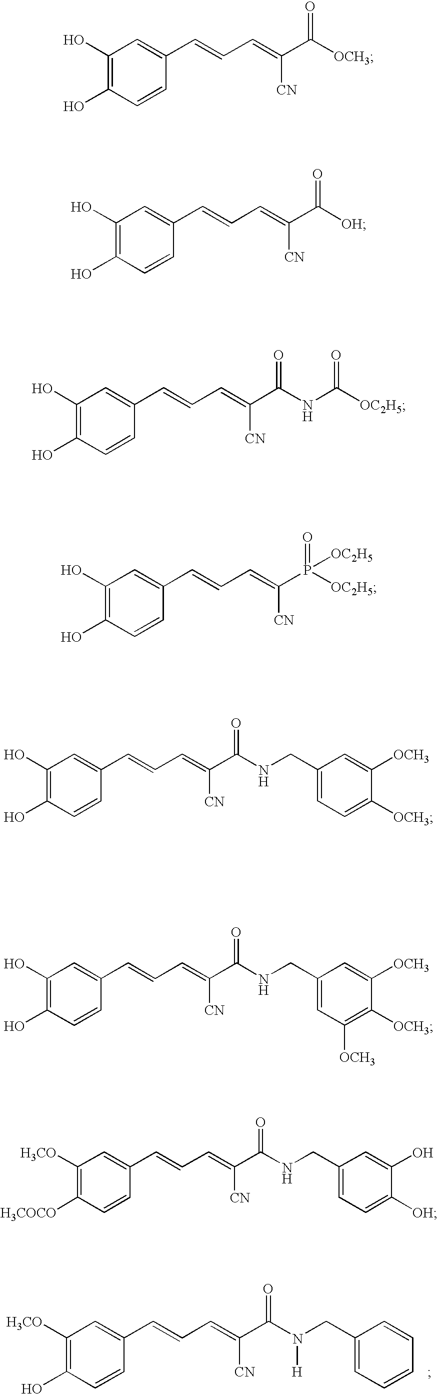

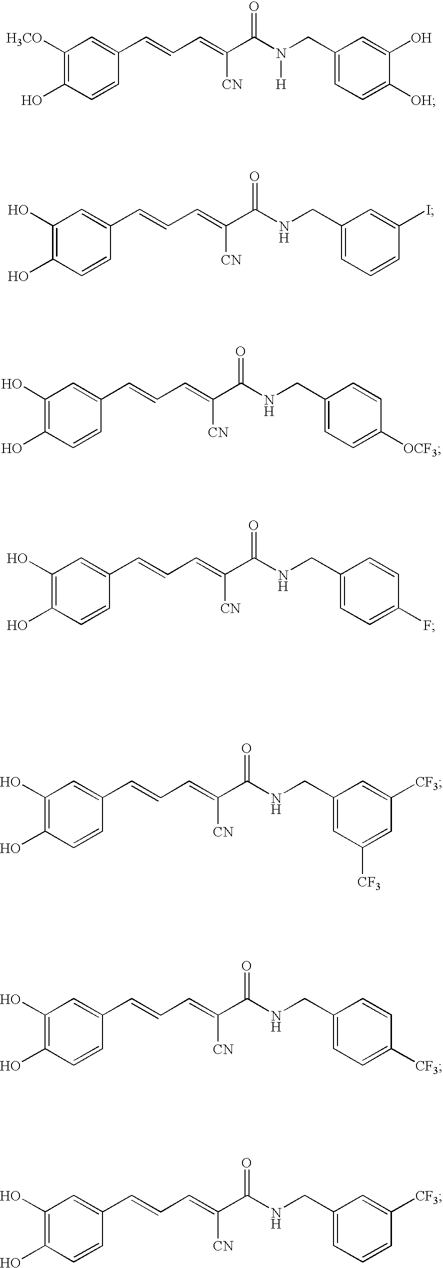

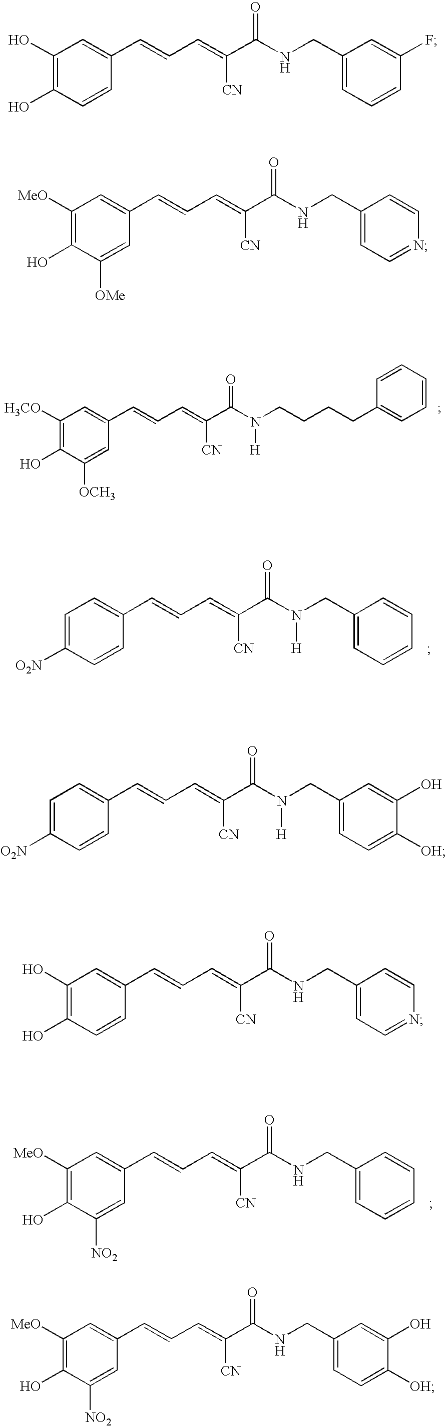

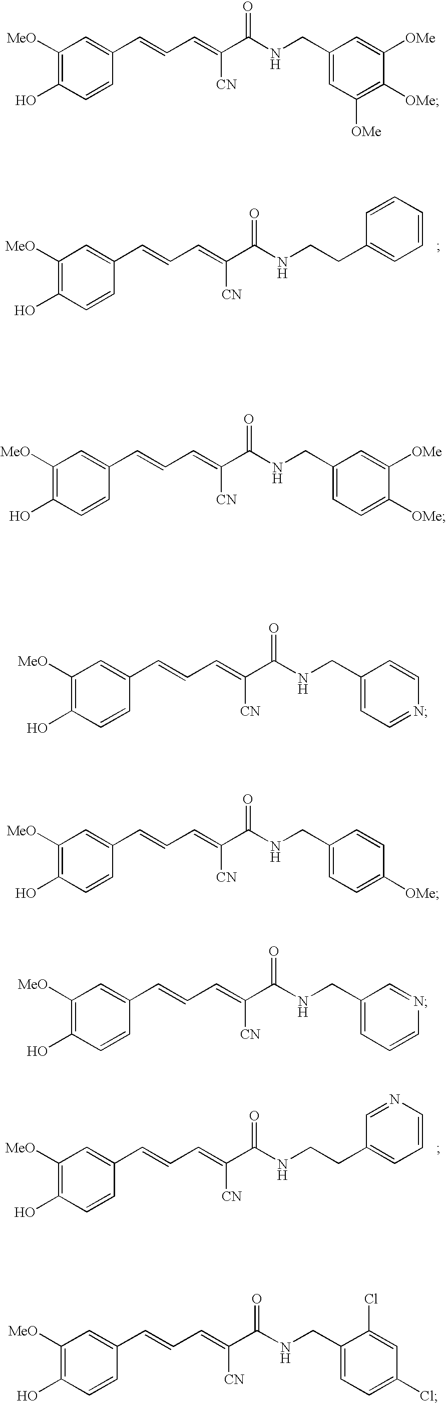

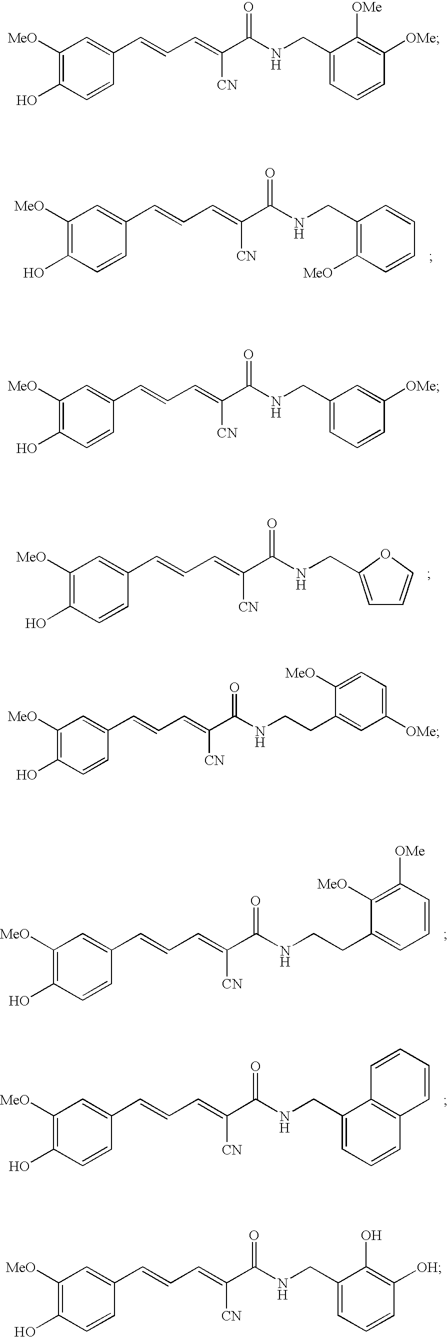

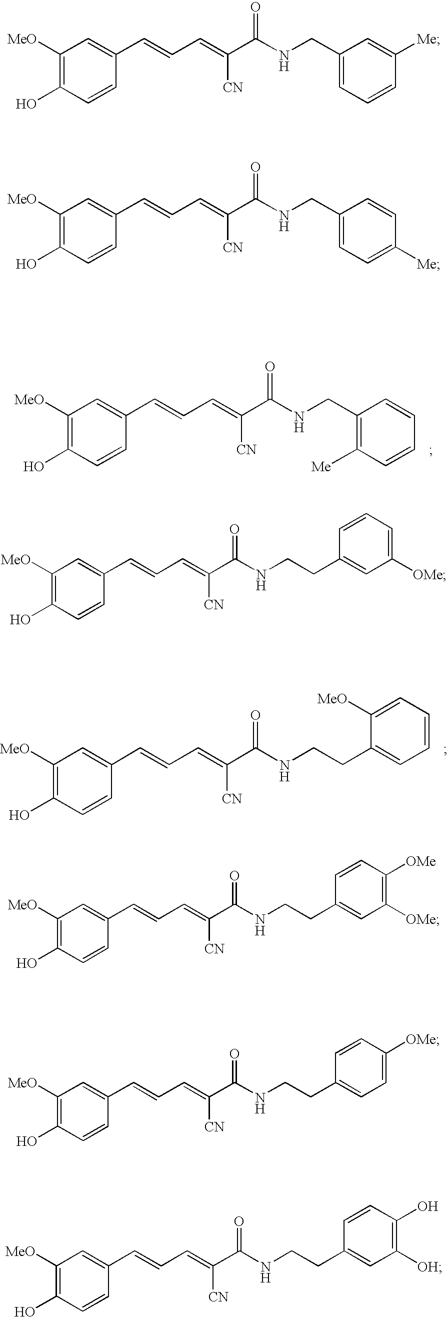

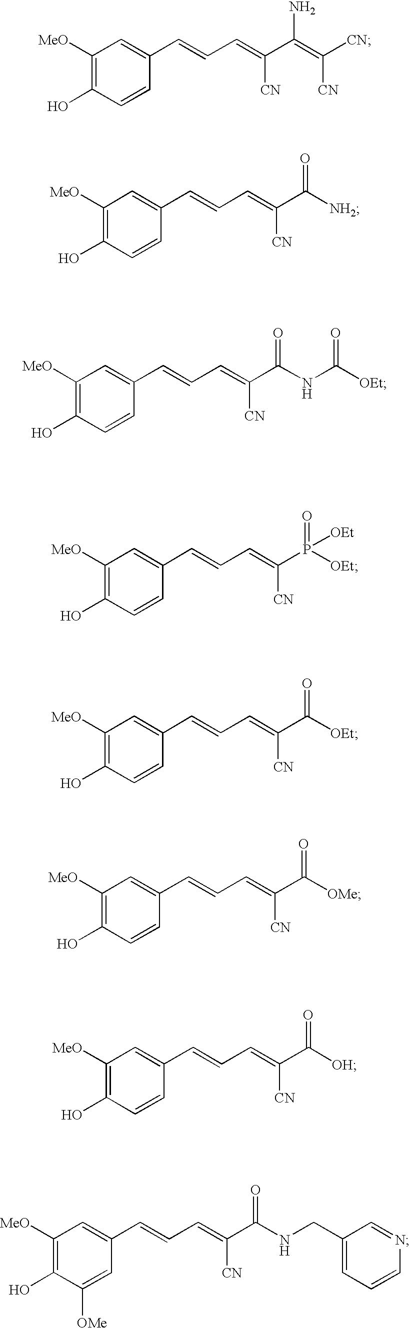

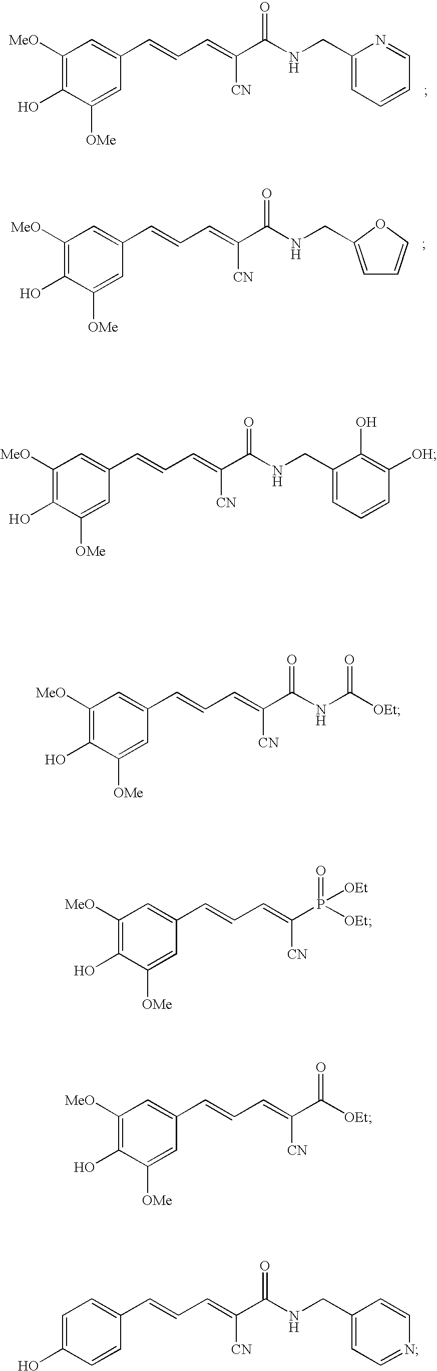

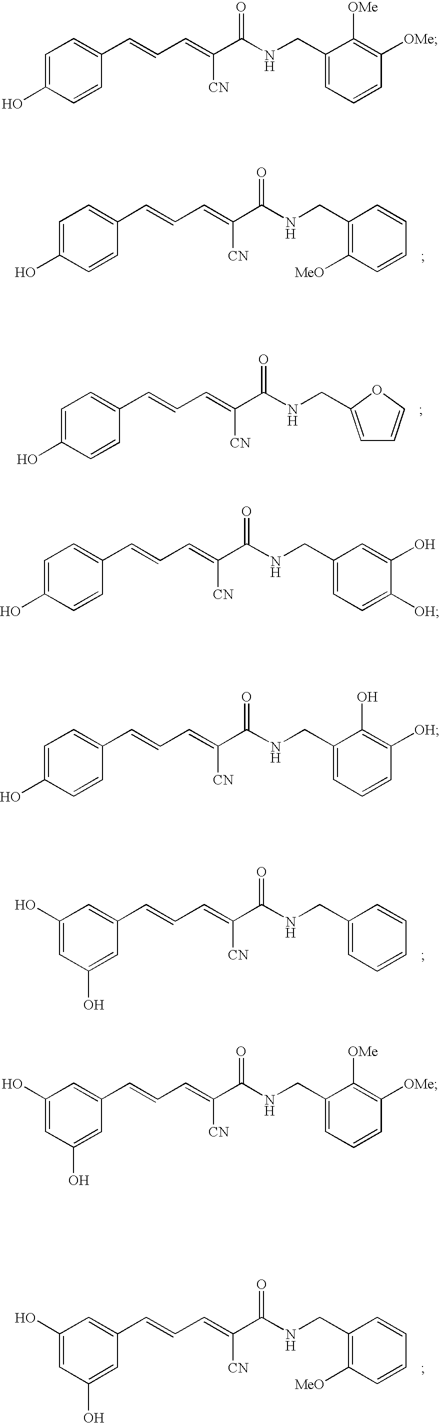

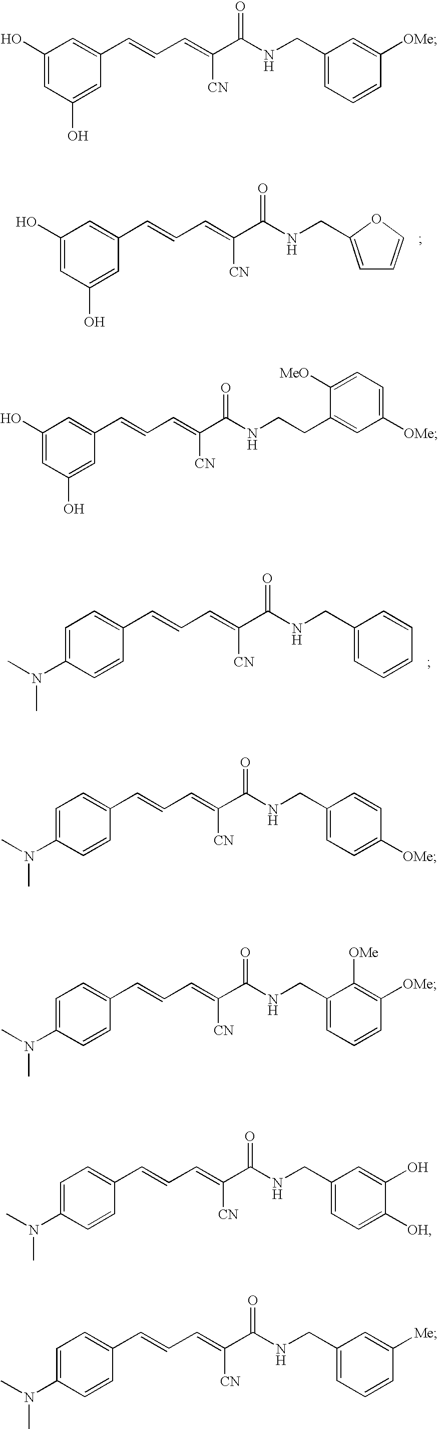

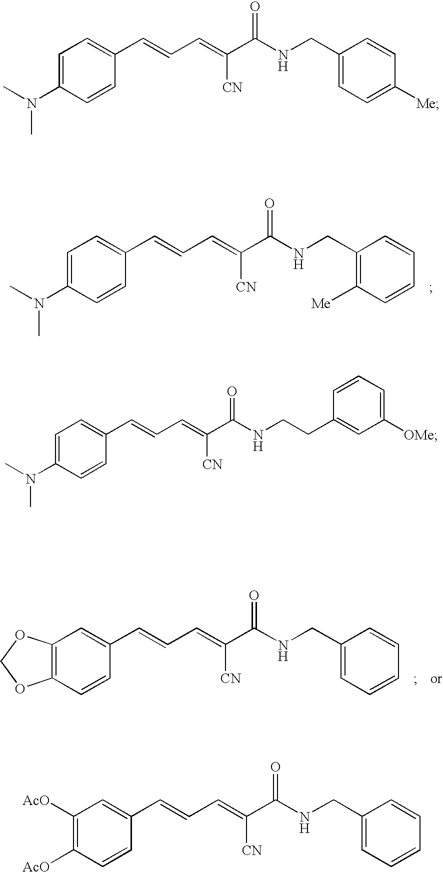

The reaction conditions for carrying out the Knoevenagel reaction are known to the person skilled in the art and also apply to the reaction according to the invention of the compounds of the general formulae (I), (VII), and (IX).

Specific solvents suitable for the purification and crystallization of the compounds of the general formula (V) and (VIII) are, for example, ethanol, dimethylformamide, ether, acetonitrile, tetrahydrofuran, dioxane, acetone, 2-butyloxyethanol, 2-ethoxyethanol, 2-isopropoxyethanol, 2-methoxyethanol, 2-propyloxyethanol, 2-butyloxyethanol, 1-methoxy-2-propanol, diethylene glycol diethyl ether, triethylene glycol monomethyl ether, triethylene glycol monomethyl ether.

IV. Exemplification

The invention now being generally described, it will be more readily understood by reference to the following examples which are included merely for purposes of illustration of certain aspects and embodiments of the present invention, and are not intended to limit the invention.

### EXAMPLE 1

Reaction of 1-bromo-3,4-methylenedihydroxybenzene with Acrolein Ethylene Acetal, Heck Reaction

(A) 28.6 g (0.270 mol) of sodium carbonate, 50.3 g (0.503 mol) of acrolein ethylene acetal, 50.3 g (0.250 mol) of 1-bromo-3,4-methylenedioxybenzene, 5.0 g (0.013 mol) of DPPE [1,2-bis(diphenylphosphino)ethane], 1.5 g (0.007 mol) of Pd(OAc)₂ and 75 mL of dimethylformamide (DMF) were initially introduced into a 750 mL sulphonation flask which had been rendered inert. The sulphonation flask was rendered inert with nitrogen, heated to 110° C. and the mixture was stirred for 23 hours at this temperature. After 23 hours, the solution was filtered hot into another 750 mL sulphonation flask. The filtrate was cooled to room temperature. At room temperature, 500 mL of toluene were added to the reaction mixture, and the solution was cooled to 4° C. in an ice bath. Since a solid had precipitated out at 4° C., the solution was filtered off and the residue (6.39 g of a pale grey, damp solid) was then washed with cold toluene. The filtrate (653.6 g of a dark brown, slightly opaque solution) was initially introduced into 1 L separating funnel and extracted with 2×80 mL of demineralized water. After the extraction, the remaining organic phase (553.6 g of a dark red, slightly opaque solution) was filtered over silica gel, and the silica gel was then washed with 2×40 mL of toluene. The filtrate (620.2 g of a pale brown, clear solution) was dried with magnesium sulphate, filtered off into a 1 L round-bottomed flask, and the residue was then washed with toluene. This solution was concentrated by evaporation to 79.0 g and admixed with 100 mL of methanol. The resulting solution was heated to reflux, stirred under reflux for 30 minutes, cooled to 0-5° C. and treated with seed crystals, whereupon crystallization started. The suspension was then stirred for a further 1-24 hours at 0-5° C. and filtered off, and the residue was washed with a small amount of cold methanol. Drying in a drying cabinet gives 35-45 g of a slightly yellowish product [trans-3-(4,5-methylenedioxyphenyl)-2-propene ethylene acetal], which was analysed by means of NMR.

(B) 4.0 g of the product obtained under preceding stage (A) and 7 ml of methanol were initially introduced into a 50 mL three-necked round-bottomed flask and heated to reflux until the crystals had completely dissolved. The solution was further heated for 30 minutes, cooled to room temperature and then further cooled to 2° C. using an ice bath. The suspension was then stirred for 2 hours and filtered off and the residue (4.2 g of slightly yellowish moist crystals) were washed with 1-2 ml of cold methanol.

The crystals obtained were dried overnight in a drying cabinet at 40° C. and about 20 mbar. Drying gave 3.7 g (yield: 93%) of slightly yellowish crystals. The purity of the crystals was confirmed by means of HPLC.

### EXAMPLE 2

Acetal Deprotection of trans-3-(4,5-methylenedioxyphenyl)-2-propene ethylene acetal From Example 1

4.0 g (15.98 mmol) of the trans-3-(4,5-methylenedioxyphenyl)-2-propene ethylene acetal (crude) obtained in stage (A) of Example 1 were initially introduced into a 100 mL round-bottomed flask and dissolved in 20 mL of tetrahydrofuran (THF). At room temperature, 45.4 mL of HCl (1 N) were added under nitrogen to the reaction mixture over the course of 45 minutes, during which crystals precipitated out. When all of the HCl had been added, the suspension was stirred for 2 hours, the suspension was filtered off and the residue (3.4 g of slightly yellow, moist crystals) was then washed with water. Drying under reduced pressure at 40° C. gave 2.7 g (yield: 85%) of product (3,4-methylenedioxycinnamaldehyde). The purity and the identity were determined by means of HPLC and ¹H NMR.

### EXAMPLE 3

Reaction of 3,4-methylenedioxycinnamaldehyde with 2-benzylamidoacrylonitrile, Knoevenagel Reaction

2.0 g (11.13 mmol) of the product from Example 2 (3,4-methylenedioxycinnamaldehyde), 76.8 g of ethanol (absolute) and 0.1093 g of piperidine were initially introduced into a 250 mL three-necked round-bottomed flask. The suspension was stirred at room temperature for 1 hour to dissolve the crystals. Then, 2.26 g of (cyanoacetyl)benzamide were added to the reaction mixture. This solution was stirred for 6-8 hours, during which a suspension was formed. After 6-8 hours, the suspension was filtered off and the residue (3.4 g of yellow, moist crystals) was washed with a small amount of absolute ethanol. This residue was dried overnight in a drying cabinet at 40° C. and about 20 mbar. Drying gave 2.7 g of (E,E)-2-(benzylamido)-3-(3,4-methylenedioxystyryl)acrylonitrile (yield: 71%) as yellow crystals. The identity and purity were confirmed by means of ¹H NMR and HPLC.

### EXAMPLE 4

Methylene Group Elimination

Under an argon atmosphere, 1 g of (E,E)-2-(benzylamido)-3-(3,4-methylenedioxystyryl)acrylonitrile was dissolved in 20 mL of dichloromethane (DCM) and cooled to an internal temperature (IT) of −20° C. Using a syringe, 5.7 mL of BBr₃ were added over the course of 5-10 minutes and the solution was firstly stirred for 1 hour at IT −20° C. and then heated to IT 15-25° C. In accordance with TLC monitoring, 10 mL of water are carefully added and the mixture is transferred to a dropping funnel and 20 mL of DCM and 2 mL of HCl (1 N) are added. The mixture is stirred for 10 minutes, the phases are separated and the aqueous phase is extracted again with 20 mL of DCM. The combined organic phases are dried over MgSO₄, and filtered off and the DCM phase is concentrated by evaporation. This gives a yellowish residue as (E,E)-2-(benzylamido)-3-(3,4-dihydroxystyryl)acrylonitrile in a yield of 70% (HPLC analysis). The resulting (E,E)-2-(benzylamide)-3-(3,4-dihydroxystyryl)acrylonitrile was then recrystallized from acetonitrile.

### EXAMPLE 5

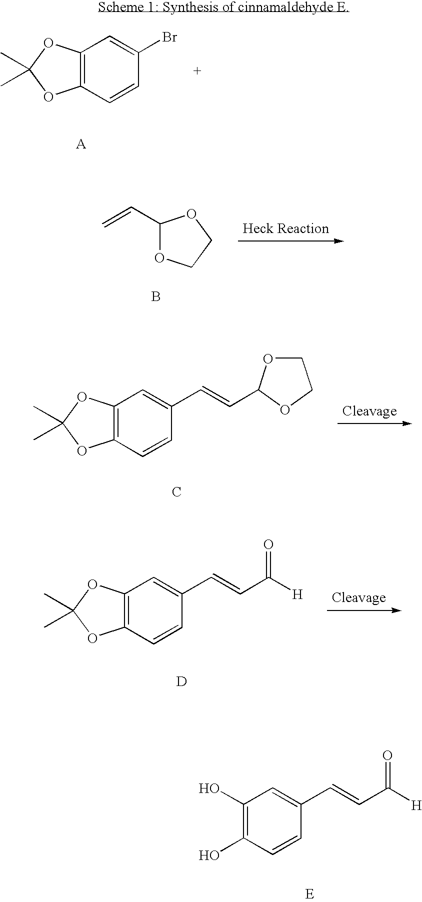 Synthesis of Intermediate C

4-Bromo-1,2-dihydroxybenzolacetonide A (160.0 g) was combined with Na₂CO₃ (72.0 g), DPPE (12.7 g), Pd(OAc)₂ (3.8 g) and acrolein ethylenacetal B (127.0 g) were suspended under an N₂ atmosphere in DMF (200.0 g). The yellow suspension was heated to 105-110° C. for 32-36 hours, at which time the suspension turned a brownish color. After 32-36 hours, an in process control (IPC) was performed, whereby if the amount of starting material less than 2% (HPLC), the suspension is cooled down to 25° C. and 320 g of ethyl acetate is added. If the amount of starting material is greater than 2%, the suspension is heated for two additional hours. The suspension was then filtered over nutsch and the residue rinsed with ethyl acetate (320.0 g). Water (640.0 g) and NaCl (19.2 g) were added and the mixture heated to 55-60° C. for 10 min. The phases were then separated and the aqueous. phase was discarded. Water (334 g) and NaCl (13.4 g) were added to the organic phase, the mixture was well agitated, and the phases were separated. The organic phase was then concentrated under vacuum to provide a brownish oil (208 g) which was used without further purification.

Synthesis of Cinnamaldehyde E.

Intermediate D was dissolved in water (544 g) and acetic acid (544 g) and heated to 100° C. for 22-24 hours. After 22-24 hours, an in process control was performed and if the amount of remaining starting material is less than 1%, the acetic acid/water mixture is distilled off under vacuum. If the amount of remaining starting material is greater than 1%, the mixture is refluxed for another 1-2 hours and the IPC is repeated. The resulting suspension was then cooled down over 1 h to between −8 and −5° C. To complete the crystallization, the suspension was stirred for at least 2 hours at that temperature. The suspension was then separated with a nutsch and the residue rinsed 2×35 g with MTBE. The wet material is then dried in a vacuum dryer at 45° C. to provide 33.5 g of E in a non-optimized yield of 33% over two steps.

### EXAMPLE 6

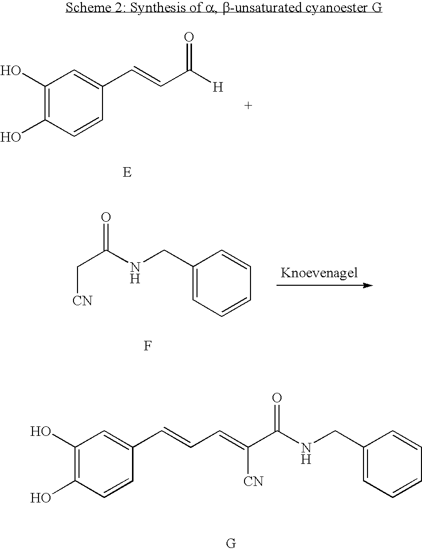 Synthesis of Cyanoester G

Cinnamaldehyde E (11.0 g; corrected with the HPLC purity) was dissolved in methanol (500 g). The solution was filtered over a nutsch, which is rinsed with methanol (80 g). At 20-25° C., intermediate F (8.5 g) was added in one portion to the methanolic solution followed by the addition of piperidine (9.1 g) in several portions. The deep red solution was stirred for at least 3 hours and an in-process control performed. Upon total conversion of starting material, 370 g of methanol was distilled off at 4045° C. To the resulting residue was added 16.6 g of HCl (32%) and water (150 g). The color changed from red to yellow and the product precipitated out of solution provided that pH was between 1 and 1.3. The suspension was then cooled to 0-5° C. and stirred for at least 2-3 hours (up to 20 hours) to maximize the yield. The suspension was then filtered via a nutsch and the residue rinsed with water (25 g) to yield 15.1 g of G. The wet G (15 g) was then suspended in acetonitrile (600 g) and heated to 80-82° C. for 1 h. The suspension was then cooled to 0-5° C. and stirred for at least 3 h. The wet material was separated with a nutsch and rinsed twice with a mixture of ethanol (10 g) and water (20 g). Final drying in the vacuum dryer at 45° C. yields 10.2 g of yellowish G in an overall yield of 56% (over two steps).

Equivalents

Those skilled in the art will recognize, or be able to ascertain using no more than routine experimentation, numerous equivalents to the compounds and methods of use thereof described herein. Such equivalents are considered to be within the scope of this invention and are covered by the following claims.

All of the patents, references, and publications cited herein are hereby incorporated by reference.

1. A process for the preparation of cinnamaldehyde compounds of the general formula (I) 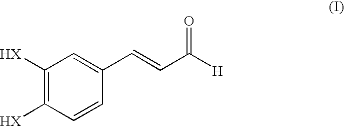
in which X is O or —NH—,
wherein a compound of the general formula (II): 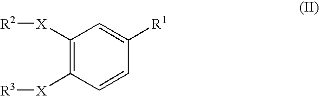
wherein
R¹ is a leaving group which is able to react in a Heck reaction as complex-forming leaving group,
X is O or —NH—; and
when X is O: R² and R³, independently of one another, are trialkylsilyl, (C₁₋₄)-allyl, (C₁₋₄)-alkenyl, aryl; or R² and R³ together are —C(CH₃)₂—, —CH₂—, —CH₂—CH₂—, —C(O)—C(O)—, or dialkylsilyl, thereby forming a ring; and
when X is —NH—, R² and R³, independently of one another, are trialkylsilyl or
alkyloxycarbonyl or phenyloxycarbonyl, or R² and R³ together are —C(O)—C(O)—;
is reacted with a compound of the general formula (III): 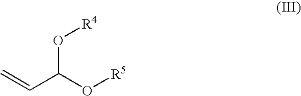
wherein
R⁴ and R⁵, independently of one another, are C₁₋₈-alkyl or trialkylsilyl; or R⁴ and R⁵ together are —CH₂—, —CH₂—CH₂— or —C(O)—C(O)—, thereby forming a ring
in a Heck reaction, and then the protective groups are removed.

2. A process according to claim 1, wherein R¹ selected from halogen, trifluoromethanesulphonate; carbonyl halide, nitro, diazo, and —N₂BF₄.

3. A process according to claim 2, wherein R¹ is selected from chlorine, bromine, iodine, trifluoromethanesulphonate, and carbonyl chloride.

4. A process according to claim 3, wherein R¹ is bromine.

5. A process according to claim 1, wherein X is O, and R² and R³, independently of one another, are trimethylsilyl or R² and R³ together are —C(CH₃)₂—, —CH₂—, or —CH₂—CH₂— or dimethylsilyl, thereby forming a ring.

6. A process according to claim 5, wherein X is O and R² and R³ together are —CH₂—, thereby forming a ring.

7. A process according to claim 1, wherein X is —NH— and R² and R³, independently of one another, are trialkylsilyl or alkyloxycarbonyl.

8. A process according to claim 7, wherein R² and R³, independently of one another, are trimethylsilyl or Boc (tert-butyloxycarbonyl).

9. A process according to claim 1, wherein R⁴ and R⁵, independently of one another, are methyl, ethyl, or trimethylsilyl, or R⁴ and R⁵ together are —CH₂—CH₂—, thereby forming a ring.

10. A process according to claim 9, wherein R4 and R⁵, independently of one another, are methyl or ethyl, or together are —CH₂—CH₂—, thereby forming a ring.

11. A process according to claim 10, wherein the compound of Formula (III) is acrolein ethylene acetal.

12. A process according to claim 1, wherein the catalyst is a compound of palladium.

13. A process according to claim 12, wherein the catalyst is selected from Pd(0) and Pd(II) compounds.

14. A process according claim 13, wherein the catalyst used is chosen from Pd(PPh₃)₄, PdCl₂, Pd(dppe)₂, Pd(dppe)Cl₂, Pd(OAc)₂, Pd(dppe)(OAc)₂, Pd(CH₃CN)₂Cl₂, Pd(PPh₃)₂Cl₂, and π-allyl-Pd complexes.

15. A process according to claim 14, wherein the catalyst is selected from π-allyl-Pd chloride dimer and tris(dibenzylideneacetone)dipalladium chloroform.

16. A process according to claim 1, wherein an additional complexing agent is added for the thermal stabilization of the palladium complex.

17. A process according to claim 16, wherein the additional complexing agent is selected from 2,2′-bipyridyl, 1,10-phenanthroline, and a phosphine compound.

18. A process for the preparation of α,β-unsaturated cyanoester and cyanoamide compounds of the general formula (IV): 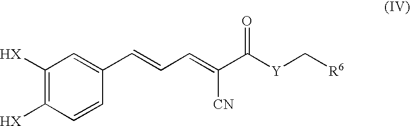
in which
Y is oxygen or —NH— and
R⁶ is optionally substituted phenyl or phenyl-(C₁₋₄)alkyl,
wherein a compound of the general formula (I) as in claim 1 is reacted in accordance with Knoevenagel conditions with a compound of the general formula (V): 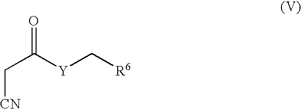
in which Y and R⁶ have the meanings given above.

19. A process according to claim 18, wherein Y is —NH— and R⁶ is phenyl.

20. A process according to claim 18, wherein one of the following compounds is prepared: (E,E)-2(benzylamido)-3-(3,4-dihydroxystyryl)acrylonitrile; (E,E)-2(phenylethylamido)-3-(3,4-dihydroxystyryl)acrylonitrile; (E,E)-2(phenylpropylamido)-3-(3,4-dihydroxystyryl)acrylonitrile; (E,E)-2(2,4-dihydroxybenzyl)-3-(3,4-dihydroxystyryl)acrylonitrile; (E,E)-2(benzylamido)-3-(3,4-diaminostyryl)acrylonitrile.

21. A process for the preparation of cinnamaldehyde compounds of the general formula (IV) 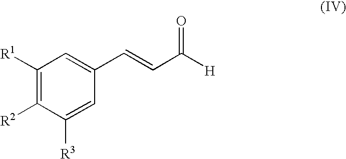
wherein
R¹ is selected from H, OH, C₁₋₆alkyl, C₁₋₆alkoxy, C₁₋₆alkylCO₂, NH₂, NH-C₁₋₆alkyl, N(C₁₋₆alkyl)(C₁₋₆alkyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, CF₃, OCF₃, and halo, or R¹ and R² together represent O-C₁₋₆alkyl-O, thereby forming a ring;
R³ is selected from H, OH, C₁₋₆alkyl, C₁₋₆alkoxy, C₁₋₆alkylCO₂, NH₂, NH-C₁₋₆alkyl, N(C₁₋₆alkyl)(C₁₋₆alkyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, halo, and CH₂—S—(CH₂)ₙ Ar;
Ar is an aromatic or heteroaromatic group, unsubstituted or substituted with 1-4 substituents, independently selected from OH, C₁₋₆alkyl, C₁₋₆alkoxy, NH₂, NH-C₁₋₆alkyl, N(C₁₋₆alkyl)(C₁₋₆alkyl), SH, S-C₁₋₆alkyl, NO₂, CF₃, OCF₃, and halo
n is 0 to 4; and
the process comprising reacting a compound of the general formula (V) 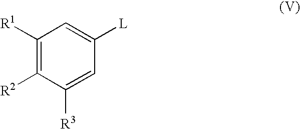
wherein
R¹ and R² are independently selected from H, C₁₋₆alkyl, C₁₋₆alkoxy, C₁₋₆alkylCO₂, NH-C₁₋₆alkyl, N(C₁₋₆alkyl)(C₁₋₆alkyl), NH(C₁₋₆alkyloxycarbonyl), NH(phenyloxycarbonyl), NH(C₁₋₆trialkylsilyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, CF₃, OCF₃ and halo, or R¹ and R² together represent O—C₆alkyl-O, thereby forming a ring;
R³ is selected from H, C₁₋₆alkyl, C₁₋₆alkoxy, C₁₋₆alkylCO₂, NH-C₁₋₆alkyl, N(C₁₋₆alkyl)(C₁₋₆alkyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, halo, and CH₂—S—(CH₂)ₙ Ar;
L is a leaving group which is able to react in a Heck reaction as complex-forming leaving group; and
Ar is an aromatic or heteroaromatic group, unsubstituted or substituted with 14 substituents, independently selected from OH, C₁₋₆alkyl, C₁₋₆alkoxy, NH₂, NH-C₁₋₆alkyl, N(C₁₋₆alkyl)(C₁₋₆alkyl), SH, S-C₁₋₆alkyl, NO₂, CF₃, OCF₃, and halo
n is 0 to 4;
with a compound of the general formula (III) 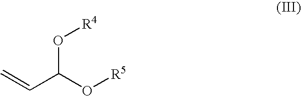
wherein
R⁴ and R⁵, independently of one another, are C₁₋₈-alkyl or trialkylsilyl; or R⁴ and R⁵ together are —CH₂—, or —CH₂—CH₂— or —C(O)—C(O)—, thereby forming a ring,
in a Heck reaction, and then the protective groups are removed.

22. A process according to claim 21, wherein R¹, R² and R³ are each independently selected from H, OH, OCH₃, CH₃CO₂, NH₂, N(CH₃)₂, and NO₂.

23. A process according to claim 22, wherein R¹, R² and R³ are each independently selected from H, OH, and OCH₃.

24. A process according to claim 21, wherein L, as leaving group, is halogen, trifluoromethanesulphonate; carbonyl halide, nitro, diazo, or —N₂BF₄;

25. A process according to claim 24, wherein L is selected from chlorine, bromine, iodine, trifluoromethanesulphonate, carbonyl chloride

26. A process according to claim 25, wherein L is bromine.

27. A process according to claim 21, wherein the catalyst is a compound of palladium.

28. A process according to claim 27, wherein the catalyst is selected from Pd(PPh₃)₄, PdCl₂, Pd(dppe)₂, Pd(dppe)Cl₂, Pd(OAc)₂, Pd(dppe)(OAc)₂, Pd(CH₃CN)₂Cl₂, Pd(PPh₃)₂Cl₂, π-allyl-Pd complexes

29. A process according to claim 28, wherein the catalyst is selected from π-allyl-Pd chloride dimer and tris(dibenzylideneacetone)dipalladium chloroform.

30. A process according to claim 21, wherein an additional complexing agent is added, wherein the complexing agent is selected from 2,2′-bipyridyl, 1,10-phenanthroline, or a phosphine compound.

31. A process for the preparation of α,β-unsaturated cyanoester and cyanoamide compounds of the general formula (VIII) 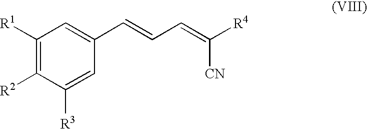
wherein
R¹ and R² are independently selected from H, OH, C₁₋₆alkyl, C₁₋₆alkoxy, C₁₋₆alkylCO₂, NH₂, NH-C₁₋₆alkyl, N(C₁₋₆alkyl)(C₁₋₆alkyl), C₁₋₆alkyl(C═O)NH, C₁₋₆alkyl(C═O)N(C₁₋₆alkyl), SH, S-C₁₋₆alkyl, O—Si(C₁₋₆alkyl)(C₁₋₆alkyl)(C₁₋₆alkyl), NO₂, CF₃, OCF₃, and halo, or R¹ and R² together represent O-C₁₋₆alkyl-O, thereby forming a ring;
R³ is selected from H, OH, C₁₋₆alkyl, C₁₋₆alkoxy, C₁₋₆alkylCO₂, NH₂, NH—C₁₋₆alkyl, N(C₁₋₆alkyl)(C₁₋₆alkyl), C₁₋₆alkyl(C═O)NH, C₁₋₆alkyl(C═O)N(C₁₋₆alkyl), SH, S-C₆alkyl, O—Si(C₁₋₆alkyl)(C₁₋₆alkyl)(C₁₋₆alkyl), NO₂, halo, and CH₂—S—(CH₂), Ar; and
R⁴ represents C(X)R⁵, SO₃Ar, SO₂Ar, SO₂(C₁₋₆alkyl), NH₂, NH-C₁₋₆alkyl, N(C₁₋₆alkyl)(C₁₋₆alkyl), P(O)(OH)₂, P(O)(OC₁₋₆alkyl)₂, and C(NH₂)═C(CN)₂;
X is selected from O, S, NH, and N-C₁₋₆alkyl;
R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, (OCH₂CH₂)ₚOCH₃, NHNH₂, NHC(O)NH₂, NHC(O)C₁₋₆alkoxy, N-morpholino, and N-pyrrolidino;
Ar is an aromatic or heteroaromatic group, unsubstituted or substituted with 1-4 substituents, independently selected from OH, C₁₋₆alkyl, C₁₋₆alkoxy, NH₂, NH-C₁₋₆alkyl, N(C₁₋₆alkyl)(C₁₋₆alkyl), SH, S-C₁₋₆alkyl, NO₂, CF₃, OCF₃, and halo;
n is 0 to 4;
p is 1 to 4; and
the process is wherein a compound of the general formula (VIII) is reacted in accordance with Knoevenagel with a compound of the general formula (IX) 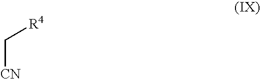
wherein
R⁴ represents C(X)R⁵, SO₃Ar, SO₂Ar, SO₂(C₁₋₆alkyl), NH₂, NH-C₁₋₆alkyl, N(C₁₋₆alkyl)(C₁₋₆alkyl), P(O)(OH)₂, P(O)(OC₁₋₆alkyl)₂, and C(NH₂)═C(CN)₂;
X is selected from O, S, NH, and N-C₁₋₆alkyl;
R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, (OCH₂CH₂)ₚOCH₃, NHNH₂, NHC(O)NH₂, NHC(O)C₁₋₆alkoxy, N-morpholino, and N-pyrrolidino;
Ar is an aromatic or heteroaromatic group, unsubstituted or substituted with 1-4 substituents, independently selected from OH, C₁₋₆alkyl, C₁₋₆alkoxy, NH₂, NH-C₁₋₆-alkyl, N(C₁₋₆alkyl)(C₁₋₆alkyl), SH, S-C₁₋₆alkyl, NO₂, CF₃, OCF₃, and halo;
n is 0 to 4; and
p is 1 to 4.

32. A process according to claim 31, wherein R¹, R² and R³ are each independently selected from H, OH, OCH₃, CH₃CO₂, NH₂, N(CH₃)₂, and NO₂.

33. A process according to claim 32, wherein R¹, R², and R³ are each independently selected from H, OH, and OCH₃ with the proviso that at least one of R¹, R², and R³ is other than hydrogen.

34. A process according to claim 31, wherein R⁴ is selected from C(X)R⁵, SO₂Ar, SO₂(C₁₋₆alkyl), and C(NH₂)═C(CN)₂.

35. A process according to claim 34, wherein R⁴ is C(X)R⁵.

36. A process according to claim 31, wherein X is O or S, R⁵ is selected from NH₂, OH, NH(CH₂)ₚAr, (CH₂)ₚOH and C₁₋₄alkoxy, and p is 1-3.

37. A process according to claim 36, wherein X is O, R⁵ is selected from NH₂, OH, NH(CH₂)ₚAr, NH(CH₂)ₚOH and OCH₃, and p is 1-2.
